(19) United States
(12) Patent Application Publication
(10) Pub. No.: US 2022/0401497 A1
(43) Pub. Date: Dec. 22, 2022

(54) USAGE OF EGGSHELL MEMBRANE FOR OBTAINING A FUNCTIONAL RAW MATERIAL AND FOR PURIFICATION OF NATURAL COMPOUNDS AS BIOADSORBENT

(71) Applicant: EGE UNIVERSITESI, Izmir (TR)

(72) Inventors: Oguz BAYRAKTAR, Izmir (TR); Feyruz BOZOGLAN, Izmir (TR)

(73) Assignee: EGE UNIVERSITESI, Izmir (TR)

(21) Appl. No.: 17/776,633

(22) PCT Filed: Nov. 10, 2020
(86) PCT No.: PCT/TR2020/051070

§ 371 Date: May 13, 2022

(30) Foreign Application Priority Data
Nov. 13, 2019 (TR) 2019/17677
Sep. 21, 2020 (TR) 2020/14981

(51) Int. Cl. A61K 35/57; A61K 36/63; A61K 31/7048; A61K 31/05; A23L 15/00; A23L 33/105; A23L 3/3463

(52) U.S. Cl. CPC A61K 35/57; A61K 36/63; A61K 31/7048; A61K 31/05; A23L 15/00; A23L 33/105; A23L 3/34635; A23V 2002/00

(57) ABSTRACT

Eggshell membranes with high collagen and hyaluronic acid content are separated from the eggshell wastes by means of a foam separation method. During the foam separation, the eggshell membranes are enriched with the extract obtained from olive leaf and olive mill wastewater, which include phenolic compounds having antioxidant and antimicrobial properties, to obtain an antimicrobial and antioxidant compound that can be used in natural supplementary food products or cosmetic products.

### CROSS REFERENCE TO THE RELATED APPLICATIONS

This application is the national phase entry of International Application No. PCT/TR2020/051070, filed on Nov. 10, 2020, which is based upon and claims priority to Turkish Patent Application No. 2020/14981, filed on Sep. 21, 2020, and Turkish Patent Application No. 2019/17677, filed on Nov. 13, 2019, the entire contents of which are incorporated herein by reference.

### TECHNICAL FIELD

The invention is related to partial or complete separation of the eggshell membrane by means of a foam separation method using eggshell waste, addition of natural compounds such as olive leaf extract or olive mill wastewater rich in phenolic compounds during said separation and therefore obtaining a composition rich in terms of antioxidants and antimicrobial agents.

The invention can be used as raw material in the pharmaceutical, cosmetic, dermocosmetic or food sectors as a compound that exhibits antioxidant or antimicrobial properties, due to the phenolic compounds that are attached to the eggshell membrane being bonded with olive leaf extract or olive mill wastewater.

### BACKGROUND

Eggshells are raw materials that are released in abundance during liquid egg production. Due to the increase in demand to pasteurized liquid eggs, eggshell production also increases and in parallel eggshell waste amounts also increase. The number of eggs produced in the year 2013 in our country, has been announced as 17 billion eggs by the Egg Production Centre Union. 30% of this amount is consumed in foreign markets and the rest is consumed domestically. Several difficulties are faced such as the costs related to the disposal of these wastes, the accessibility of disposal sites and environmental problems.

Every year thousands of tons of eggshells are labelled as waste, and some of these are sold to the feed and fertilizer sectors. The remaining waste is left as garbage to the environment. Studies relating to the use of eggshell membranes in the printing, paint and textile industry as a biosorbent for the purpose of cleaning wastewater from dyestuffs, recovering heavy metal ions and recovering silver that is used in photography bath solutions are available in the prior art.

As it is known, eggshells are waste that should be recycled and utilized as they contain valuable substances in their composition. Although recycling of eggshell waste is significantly important in terms of environmental factors, it is important in terms of economy to convert eggshell waste into products with high added value such as food additives, feed additives and cosmetic raw materials.

In general, when natural compounds are subjected to environmental conditions such as light, heat, etc. in liquid environments, reduction is observed in their stability and in turn, in their bioactivities. Natural compounds are found in the environment in low purity. Purifying and isolating natural compounds from extracts or food industry wastes, especially those found in plants and those containing several natural compounds, is both a difficult and expensive process. In the prior art, relatively expensive resin and synthetic polymers are used as adsorbents in the purification applications of these natural compounds, and the monomers released during the use of these synthetic adsorbents pose a problem because they are toxic.

For these reasons, it is necessary to develop cheap, biocompatible and biodegradable bioadsorbents and also to develop new multifunctional value-added products with the use of these bioadsorbents.

### SUMMARY

The invention is related to obtaining a value added antioxidant and antibacterial composition by, the partial or complete separation of the membrane from the eggshell that is classified as waste material that is to be used as animal feed or fertilizer and by, enriching the separated membrane with olive leaf extract and phenolic compounds that are available in the olive mill wastewater.

The eggshell wastes that are formed as food waste are utilized by means of the invention. By means of the foam separation method the membrane of the eggshell is partially or completely separated from the shell and the separated membrane is used as a bioadsorbent and bioactive natural compounds are loaded thereon selectively, and therefore an antimicrobial and antioxidant compound is obtained that can be used in natural supplementary food products or cosmetic products.

The antimicrobial and antioxidant composition obtained by means of the invention can additionally be used as feed additive or for the preparation of medicines.

The eggshell membrane used in the invention is relatively cheap and has both biocompatible and degradable properties in its protein structure and therefore it is highly suitable to be used in health and food applications.

The stabilities and therefore the bioactivities of natural compounds in comparison to the liquid medium is maintained by means of the adsorption of natural compounds to solid surfaces having high protein content such as eggshell membranes. It is also possible to purify natural compound or compounds that are selectively adsorbed by desorption with solvents having suitable polarity.

Within this context, by means of the foam separation method that is developed from eggshell waste that poses significant economical and environmental importance, it is enabled to obtain a functional, high added value composition by separating the shell and membranes from each other without being harmful to the environment and without creating any side waste, and by adsorbing the phenolic compounds obtained from the olive mill wastewater and olive leaf extract that are added concurrently.

By the invention the concurrent separation and functionalization of the eggshell membrane is provided using the foam separation method, without using alcohol or any harmful chemical agents. The eggshell membrane that comprises collagen that has hydrophobic properties, can be used as a suitable carrier system and bioadsorbent. Natural compounds that are adsorbed on the eggshell membrane, are subjected to desorption with suitable solvents and they are used as bioadsorbents for the first time in purification processes. The compounds that are bound to the eggshell membrane are kept in a solvent that is selected according to natural compounds therein and according to the properties of the extract that is delivered to the system, and the compounds that have bound onto the surface of the membrane are passed onto the solvent phase. As a result the desired natural compounds are purified and high added value products are obtained. The purification method has been carried out to increase the quality of the end product that is desired to be obtained.

By means of the invention, the membrane with high collagen and hyaluronic acid content, which will be obtained from the eggshell, is enriched with the extract obtained from olive leaf and olive mill wastewater comprising a phenolic compound having antioxidant and antimicrobial properties, which is also a regional agricultural waste, as a result a new functional and high added value composition that can be used in the food, feed, cosmetic and pharmaceutical sector is obtained.

During the foam separation method that is developed for natural compounds such as oleuropein and hydroxythyrosol that are present in olive leaves and olive mill wastewater that are agricultural waste, when the membrane is being separated from the shell, these compounds need to be adsorbed to the surface of the membrane selectively and afterwards this membrane needs to be fractioned with solvents having different polarities (for example water-ethanol solvents having different concentrations) and these compounds need to be purified. This makes it possible to economically develop new bioadsorbents as an alternative to separation-purification processes where monomer transition to products made with synthetic resins has some negative health effects.

It is possible to use the bioactive natural compounds within the olive leaf extract and the olive mill wastewater as bioadsorbents by being loaded selectively on the eggshell membrane and as raw materials in supplementary food products or cosmetics.

By means of the invention the possibilities of the pure natural compounds obtained by separation through desorption of natural compounds that are loaded on the eggshell membrane, to be used in different industrial applications where high purity is sought for their usage in the formulation of different products such as pharmaceutical industry shall be increased. As a result, the added value of the natural compounds that are obtained as pure compounds shall also be increased. The most important reason for purification of natural compounds after being separated from the eggshell membrane is that these natural compounds can be sold in the market for a higher price.

With the invention, the potential of the eggshell membrane to selectively adsorb polyphenols within the extract obtained from olive leaves and olive mill wastewater, which are agricultural and food industry wastes, was determined. As a result of this invention, an antioxidant and antimicrobial compound that is edible, made of eggshell membranes that can selectively adsorb the natural compounds found in the olive leaf extract and the olivemill wastewater is obtained.

The aim of the invention is to separate the eggshell membrane from the eggshells that constitute an important waste problem in the food sector and to enrich the composition with natural compounds having high antioxidant and antimicrobial properties that shall be added during the separation process. As a result, the waste that is abundant following the production of pasteurized liquid eggs, and olive leaves and olive mill wastewater that are also qualified as food and agricultural waste can also be utilized. Moreover, the addition of extra functionality through adsorption by using the hydrophobic interaction property of the eggshell membranes that are to be obtained, and the addition of a plant extract that is rich in antioxidants helps to enrich the functionality of the additive agent. By using an agent with a plurality of functions in the food and cosmetics industry, a serious contribution will be made to reduce cost rates.

It is possible to obtain new raw materials by adsorption and isolation onto the eggshell membrane of the natural compounds that are to be obtained from waste via hydrophobic interaction and moreover with the concurrent usage thereof. Through the economical and multi-functional uses of these new raw materials, their usage potential in the food, cosmetic and pharmaceutical industries has been increasing. Therefore, the combination of the eggshell membrane, which contains amino acids beneficial for health, with high-capacity antioxidants can be used as a functional food and as food supplements.

### BRIEF DESCRIPTION OF THE DRAWINGS

FIG. 1: Process diagram of the foam separation (fractionalization) method for separating eggshell membranes.

FIG. 2: Foam separation (fractionalization) apparatus

FIG. 3: FT-IR graphic of the eggshell membrane and the eggshell membrane that has adsorbed olive leaf extract A) Eggshell membrane FT-IR spectrum B) FT-IR spectrum of the eggshell membrane after adsorption.

FIG. 4: Scanned electron microscope (SEM) view of the eggshell membrane.

FIG. 5: Scanned electron microscope (SEM) view of the eggshell membrane that has adsorbed the olive leaf extract.

FIG. 6: High performance liquid chromatography (HPLC) profiles of olive leaf extracts (OLE) having two different concentrations A: 3% OLE, weight/volume); B: 2% OLE, weight/volume.

FIGS. 7A-7G: The 3-dimensional response surface graphics of the change of the total antioxidant capacity with parameters. FIG. 7A: The 3-dimensional response surface graphics of the change of the total antioxidant capacity with ethanol concentration (%) and liquid/solid ratio (S/K) parameters. FIG. 7B: The 3-dimensional response surface graphics of the change of the total antioxidant capacity with ethanol concentration (%) with extraction time (hour) parameters. FIG. 7C: The 3-dimensional response surface graphics of the change of the total antioxidant capacity liquid/solid ratio (S/K) parameters and extraction time (hour) parameters. FIG. 7D: The graph of the percentage proximity of the test results to the predicted values; FIG. 7E: The graph of the percentage proximity of the test results to the test results; FIG. 7F: Histogram graphic of the test results. FIG. 7G: Random distribution of the tests.

FIGS. 8A-8G: The 3-dimensional response surface graphics of the change of the total phenol amount with parameters. FIG. 8A: The 3-dimensional response surface graphics of the change of the total phenol capacity with ethanol concentration (%) with extraction time (hour) parameters.

FIG. 8B: The 3-dimensional response surface graphics of the change of the total phenol capacity with ethanol concentration (%) and liquid/solid ratio (S/K) parameters. FIG. 8C: The 3-dimensional response surface graphics of the change of the total phenol capacity liquid/solid ratio (S/K) parameters and extraction time (hour) parameters. FIG. 8D: The graph of the percentage proximity of the test results to the predicted values; FIG. 8E: The graph of the percentage proximity of the test results to the test results; FIG. 8F: Histogram graphic of the test results. FIG. 8G: Random distribution of the tests.

### DEFINITIONS OF THE PARTS/ASPECTS/SECTIONS FORMING THE INVENTION

7. Collecting the waste eggshells.
8. Sterilizing the eggshells with water and/or acetic acid.
9. Milling of the sterilized shells
10. Delivering the milled shell into the foam separation (column)
11. Delivering air through the air inlet to the feed unit
12. Adding olive leaf extract or olive mill wastewater into the column
A: The antioxidant and antimicrobial composition of the invention
B: Additional product which is eggshells with high calcium content that remains under
the column.
C: Air
D: Feeding unit
E: Stainless steel frit
F: Foam
G: Foam collector
H: Slaked foam

### DETAILED DESCRIPTION OF THE EMBODIMENTS

The invention is related to an antioxidant and antibacterial compound comprising eggshell membranes with adsorbed phenolic compounds available in the olive leaf extract and the olive mill wastewater.

The antioxidant and antibacterial composition of the invention comprises 99% eggshell membrane and 1% phenolic compound by weight.

In the invention eggshell wastes having high collagen content and hyaluronic acid that constitute a significant waste problem in the food sector are used, the membrane of the eggshell is partially or completely separated by means of a foam separation method and during this separation process natural compounds such as olive leaf extract or olive mill wastewater with high antioxidant and antimicrobial properties are added and as a result a compound rich in antioxidants and antimicrobial agents are obtained.

When the eggshell and the membrane is being separated herbal natural compounds that are available in the herbal extract are concurrently and are selectively adsorbed on the eggshell membrane. Eggshell membranes are given functionality due to the adsorbed natural compounds, and they gain antioxidant and antimicrobial properties.

The eggshell membrane is used as a bioadsorbent for the purification of natural compounds that are functionalized with solvents having suitable polarity, where said natural compounds are selectively adsorbed onto said membrane from an herbal extract.

A foam separation unit (column) is used to separate the eggshell and the membrane from each other. Both separation and purification processes are carried out concurrently in the column. The process for obtaining antioxidant and antimicrobial compositions from eggshell wastes is carried out at room temperature.

The method of obtaining the antioxidant and antimicrobial composition of the invention comprises the process steps of:
Collecting the waste eggshells.
Sterilizing the eggshell wastes by washing them in a water and/or acid mixture,
Milling the sterilized shells to a range of 500-5000 microns, preferably until they reach the 2000 micron range size,
Delivering the milled shells into the foam separation apparatus until they fill up 25-35% of the column and adding water and acetic acid mixture thereon,
Mixing the milled eggshell with the added acetic acid and water solvent for 1 to 5 hours,
Delivering air to the foam separation apparatus at the end of the mixing process,
Adding olive leaf extract or olive mill wastewater such that they shall constitute 70-75% of the column volume during the delivery of air,
Obtaining a foam comprising the phenolic compounds in the olive leaf extract or the olive mill wastewater that have bound onto the surface of the membrane that has been separated from the eggshell,
Collecting the obtained foam,
Removing the residual agents that have not bonded to the surface of the membrane by purification with ethanol-water mixture which is a different polarity solvent pair of the collected foam,
Leaving the foam to rest and removing the air therein, and obtaining an antioxidant and antimicrobial composition (A).

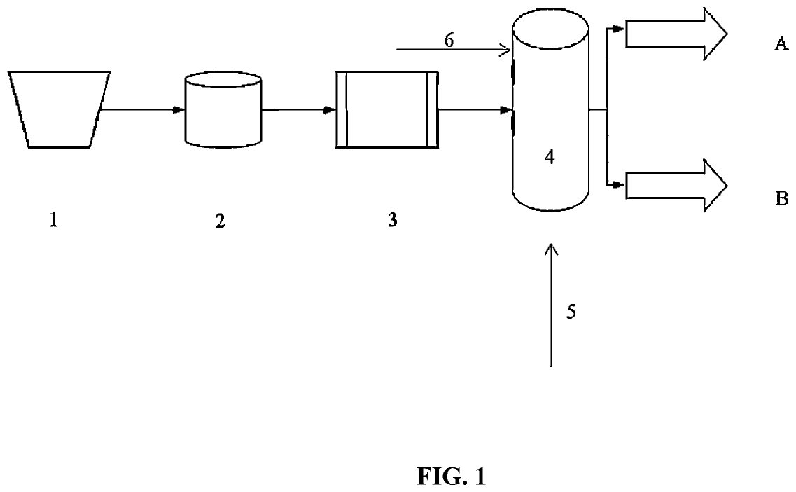

A preferred embodiment of the invention is illustrated in FIG. 1. As shown in FIG. 1, in the foam fractionalization of the eggshell membrane first of all the eggshell wastes are collected from pasteurized liquid egg producers. After the eggshells are washed with a mixture of water and 1-2% acetic acid by volume, sterilization is carried out under high pressure with vapour. The obtained sterile shells are milled with a mill. The aim of this process is to bring the egg waste to 500-5000 microns, preferably to 2000 microns and to ensure that the shell-membrane separation reaches optimum level.

The milled material is delivered to the foam separation apparatus (column). Water and acetic acid mixture is added to the eggshells in the column. In order for the membrane in the eggshell to be separated from the shell in liquid, mechanical mixing is carried out for 1 to 5 hours, preferably for 1 hour. At the end of this time, air is delivered through air inlet to the feed unit. After the addition of air the partial or complete separation of the eggshell membrane from the eggshell is started. Concurrently to the delivery of air to the feed unit, the olive leaf extract and olive mill wastewater are added to column. As a result, the antioxidant and antimicrobial composition (A) that is rich in collagen and the additive product (B) which is eggshells having high calcium content, that remain below the column following the foaming process, due to its specific weight are obtained.

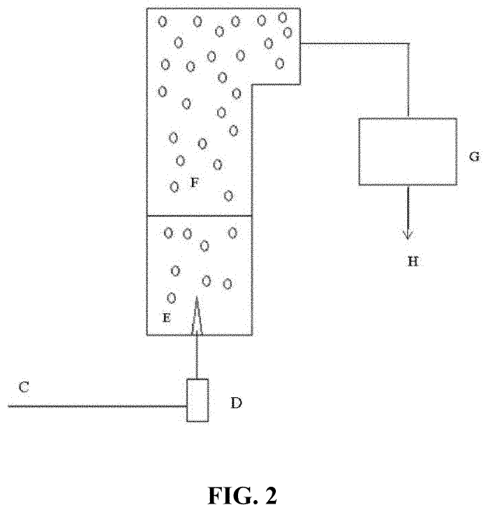

Foam fractionalization (Separation) apparatus is illustrated in FIG. 2. In the foam fractionalization apparatus, air (C) is delivered to the feed unit (D) that comprises the eggshell membrane comprising collagen, olive leaf extract and olive mill wastewater, and foaming (F) is established, and the obtained foam (F) is collected in a separate container with a foam collector (G), preferably with a separation funnel. The apparatus also contains a stainless steel frit (E) that enables the air (C) to be filtered and delivered homogenously to the column. Slaked foam (H) is obtained by leaving the air in the foam to wait and then removing it. The slaked foam (H) comprises olive leaf extract and olive mill wastewater that are carried together with the eggshell membrane loaded with the natural compound and the air foam.

In order to functionalize the membrane with collagen content, initially olive leaf extract and olive mill wastewater are added. The phenolic compounds in olive leaf extract and olive mill wastewater are adsorbed onto the membrane to obtain a functional high added value antioxidant and antimicrobial composition.

The eggshell membrane provides an ideal adsorption surface due to its natural porous and fibril structure. At the same time, the hydrophobic interaction of the collagen found in the eggshell membrane with phenolic compounds also increases adsorption.

After the membrane and the herbal extract within the foam are collected, they are used for further analysis. At this stage, the yield and the separation efficiency of the membrane is optimized by changing the parameters such as the amount of the waste eggshells and membranes, the ratios of the acetic acid mixture added to the milled eggshell, mixing time and speed, and air flow. The test design prepared for optimization has been given in Table 1. After the milled shells are transferred to the foam separation apparatus, the tests have been carried out by adjusting the water and acetic acid concentration to 0-100% (v/v), (the acetic acid concentration (0% (v/v) indicates that 100% pure water has been added) the average particle size of the eggshell to 0.5-5 mm, the mixing time to 1-5 hours, the linear flow rate of the air in column to 0.1-0.5 cm/s.

As a result of the tests carried out within the scope of the invention, it has been observed that the highest eggshell-membrane separation percentage was 81%, and this was obtained when the acetic acid concentration was 100%, the eggshell average particle size was 2 mm, and the mixing time was 5 hours, and the linear flow rate of the air in the column was 0.5 cm/s.

TABLE 1
Test design and results thereof for the separation
of the eggshell from the eggshell membrane

| | | Average | Mixing | | |
|---|---|---|---|---|---|
| | | particle | time (hour) | Linear flow | membrane-shell |
| | | size (mm) | (time kept | rate of air in | separation |
| Test | Ascetic acid | of the | in the | the column | efficiency, |
| Number | concentration %(v/v) | shell | column) | (cm/s) | percentage |
| 1 | 0 | 0.5 | 1 | 0.1 | 45 |
| 2 | 100 | 0.5 | 1 | 0.1 | 66 |
| 3 | 0 | 2 | 1 | 0.1 | 38 |
| 4 | 100 | 2 | 1 | 0.1 | 68 |
| 5 | 0 | 0.5 | 5 | 0.1 | 42 |
| 6 | 100 | 0.5 | 5 | 0.1 | 77 |
| 7 | 0 | 2 | 5 | 0.1 | 36 |
| 8 | 100 | 2 | 5 | 0.1 | 65 |
| 9 | 0 | 0.5 | 1 | 0.5 | 40 |
| 10 | 100 | 0.5 | 1 | 0.5 | 69 |
| 11 | 0 | 5 | 1 | 0.5 | 38 |
| 12 | 100 | 5 | 1 | 0.5 | 71 |
| 13 | 0 | 0.5 | 5 | 0.5 | 42 |
| 14 | 100 | 0.5 | 5 | 0.5 | 70 |
| 15 | 0 | 2 | 5 | 0.5 | 38 |
| 16 | 100 | 2 | 5 | 0.5 | 81 |
| 17 | 0 | 1.25 | 3 | 0.3 | 34 |
| 18 | 100 | 1.25 | 3 | 0.3 | 74 |
| 19 | 50 | 0.5 | 3 | 0.3 | 43 |
| 20 | 50 | 2 | 3 | 0.3 | 47 |
| 21 | 50 | 1.25 | 1 | 0.3 | 45 |
| 22 | 50 | 1.25 | 5 | 0.3 | 56 |
| 23 | 50 | 1.25 | 3 | 0.1 | 44 |
| 24 | 50 | 1.25 | 3 | 0.5 | 40 |
| 25 | 50 | 1.25 | 3 | 0.3 | 47 |
| 26 | 50 | 1.25 | 3 | 0.3 | 34 |
| 27 | 50 | 1.25 | 3 | 0.3 | 45 |
| 28 | 50 | 1.25 | 3 | 0.3 | 46 |
| 29 | 50 | 1.25 | 3 | 0.3 | 47 |
| 30 | 50 | 1.25 | 3 | 0.3 | 38 |
| 31 | 50 | 1.25 | 3 | 0.3 | 40 |

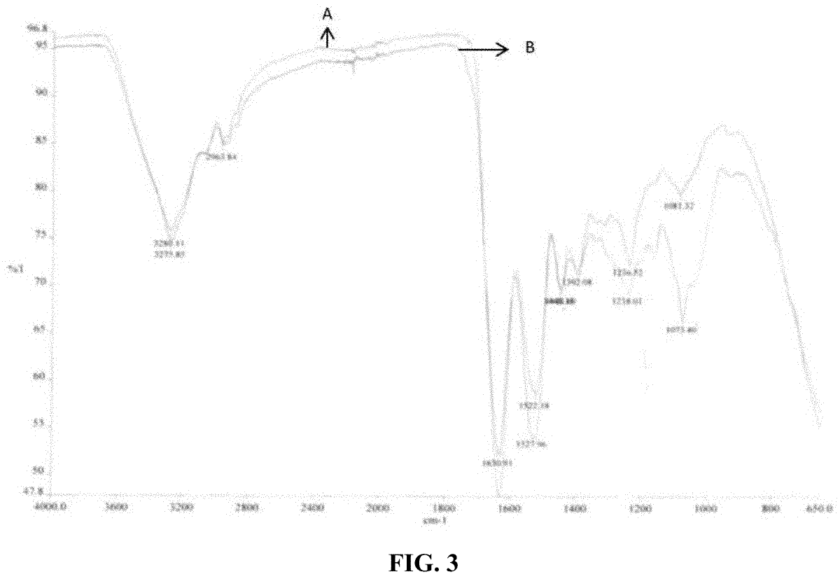

The FTIR analysis of the eggshell samples that comprise collagen and that are obtained in Table 1, is illustrated in FIG. 3, where the eggshell membrane is shown for structural analysis respectively, as the eggshell membrane and the eggshell membrane that has adsorbed olive leaf extract.

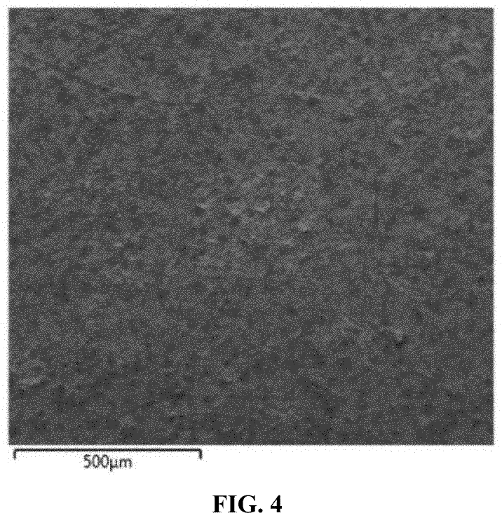

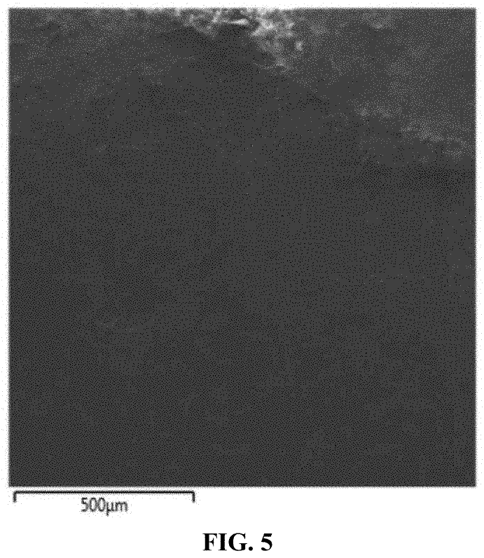

The scanned electron microscope (SEM) view of eggshell membrane is shown in FIGS. 4 and 5, respectively as the eggshell membrane and the eggshell membrane that has adsorbed olive leaf extract.

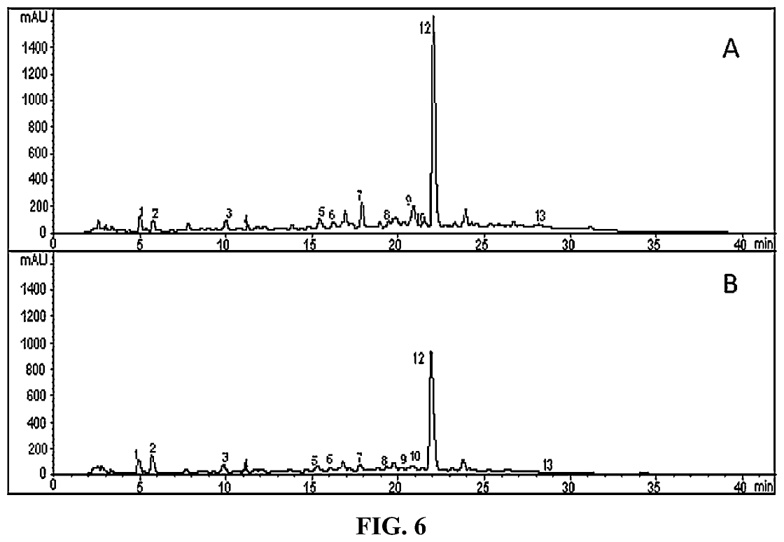

The high performance liquid chromatography (HPLC) profiles of olive leaf extract (OLE) having two different concentrations are shown in FIG. 6. The phenolic compounds that correspond to each peak number in FIG. 6 have been given in Table 2.

TABLE 2
The phenolic compounds that correspond
to each peak number in FIG. 6

| Phenolic compounds | Peak Number |
|---|---|
| Hydroxythyrosol | 1 |
| Thyrosol | 2 |
| Catechin | 3 |
| Cafeic acid | 4 |
| Vanillic acid | 5 |
| Vanillin acid | 6 |
| Rutin | 7 |
| Luteolin 7-glucoside | 8 |
| Verbascoside | 9 |
| Apigenin 7-glucoside | 10 |
| Diosmetin 7-glucoside | 11 |
| Oleuropein | 12 |
| Luteolin | 13 |

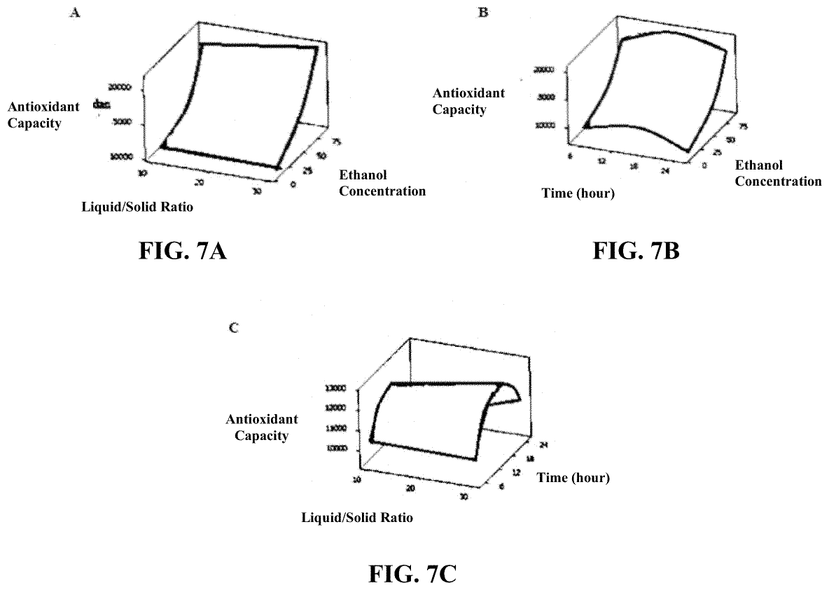

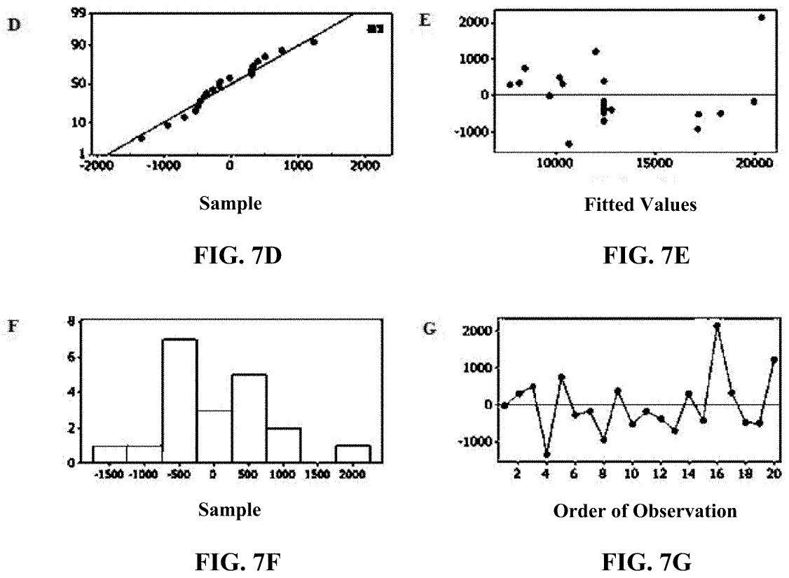

The change of the total antioxidant capacity with parameters (solid-liquid ratio, ethanol water mixture concentration and extraction time) is shown in FIGS. 7A-7G. As a result of the surface response analyses, the maximum total phenol amount has been obtained by the extraction process carried out in the 70% ethanol water mixture having 1:20 solid-liquid ratio as extraction conditions.

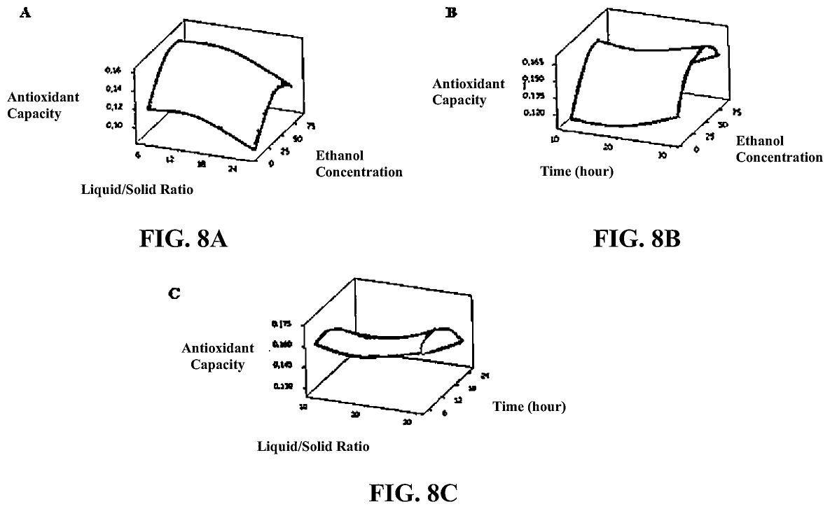

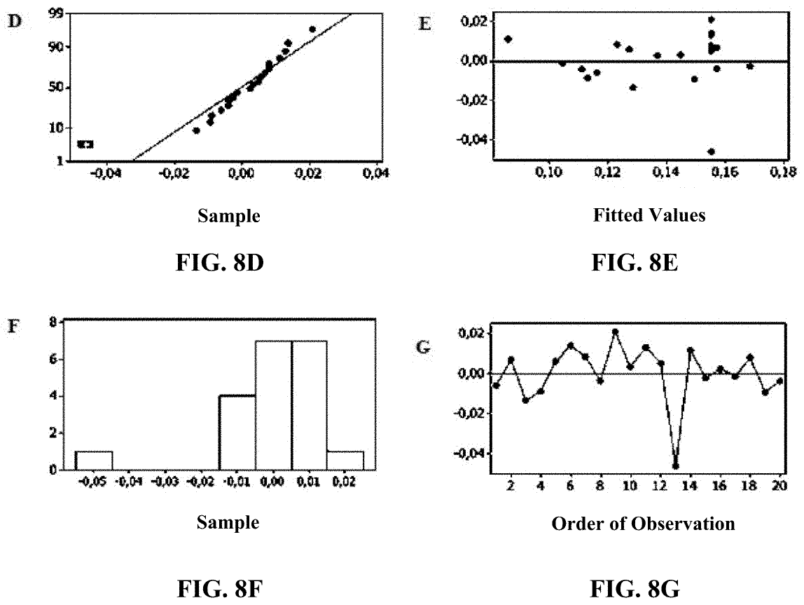

The change of the total phenol amount with parameters (solid-liquid ratio, ethanol water mixture concentration and extraction time) is shown in FIGS. 8A-8G. As a result of the surface response analyses, the maximum total phenol amount has been obtained by the extraction process carried out in the 70% ethanol water mixture having 1:20 solid-liquid ratio as extraction conditions.

What we claimed is:

1. An antioxidant and antibacterial composition, comprising egg shell membranes with adsorbed phenolic compounds, wherein the adsorbed phenolic compounds are obtained from an olive leaf extract and the olive mill wastewater.

2. The antioxidant and antibacterial composition according to claim 1, comprising 99% by weight of the eggshell membranes, and 1% by weight of the adsorbed phenolic compounds.

3. the The antioxidant and antibacterial composition according to claim 1, wherein the adsorbed phenolic compounds are Oleuropein and Hydroxythyrosol.

4. The antioxidant and antibacterial composition according to claim 1, wherein the antioxidant and antibacterial composition is configured as a supplement in natural food products or as raw materials in cosmetic products.

5. The antioxidant and antibacterial composition according to claim 1, wherein the antioxidant and antibacterial composition is configured as a feed additive or for the preparation of pharmaceuticals.

6. A production method of the antioxidant and antibacterial composition according to claim 1, comprising the process steps of:
sterilizing collected eggshell wastes by washing the collected eggshell wastes in a water and/or acid mixture to obtain sterilized eggshells,
milling the sterilized eggshells to obtain milled eggshells having a size range of 500-5000 microns,
delivering the milled eggshells into a foam separation apparatus until the milled eggshells fill up 25-35% of a column of the foam separation apparatus and adding a water and acetic acid mixture thereon, and mixing for 1 to 5 hours,
delivering air to the foam separation apparatus at an end of the mixing process,
adding the olive leaf extract or the olive mill wastewater such that the olive leaf extract or the olive mill wastewater constitutes 70-75% volume of the column during the delivery of the air,
obtaining and collecting a foam comprising eggshell membranes separated from the milled eggshells and the adsorbed phenolic compounds in the olive leaf extract or the olive mill wastewater, wherein the adsorbed phenolic compounds bind onto a surface of the eggshell membranes,
removing residual agents not bonded to the surface of the eggshell membranes by purification with an ethanol-water mixture, wherein the ethanol-water mixture is a different polarity solvent pair of the collected foam,
leaving the foam to rest and removing the air therein, and obtaining the antioxidant and antimicrobial composition.

7. The production method according to claim 6, wherein a linear velocity of the air in the foam separation apparatus is 0.1-0.5 cm/s.

8. The production method according to claim 6, wherein a linear velocity of the air in the foam separation apparatus is 0.5 cm/s.

9. The production method according to claim 6, wherein a mixing time in the foam separation apparatus is 1-5 hours.

10. The production method according to claim 6, wherein a mixing time in the foam separation apparatus is 5 hours.

11. The production method according to claim 6, wherein the sterilized eggshells are milled until the milled eggshells are 2000 microns.

12. The antioxidant and antibacterial composition according to claim 2, wherein the adsorbed phenolic compounds are Oleuropein and Hydroxythyrosol.
